(19) United States
(12) Patent Application Publication
(10) Pub. No.: US 2014/0087369 A1
(43) Pub. Date: Mar. 27, 2014

(54) INFECTION DETECTION METHODS AND SYSTEMS AND RELATED COMPOUNDS AND COMPOSITIONS

(71) Applicants: Jurgen G. Schmidt, Los Alamos, NM (US); Mark Wolfenden, Los Alamos, NM (US); Aaron Anderson, Los Alamos, NM (US); Jennifer Foster Harris, Los Alamos, NM (US)

(72) Inventors: Jurgen G. Schmidt, Los Alamos, NM (US); Mark Wolfenden, Los Alamos, NM (US); Aaron Anderson, Los Alamos, NM (US); Jennifer Foster Harris, Los Alamos, NM (US)

(73) Assignee: Los Alamos National Security, LLC, Los Alamos, NM (US)

(21) Appl. No.: 14/092,609

(22) Filed: Nov. 27, 2013

Related U.S. Application Data
(62) Division of application No. 13/294,966, filed on Nov. 11, 2011, now abandoned.
Application No. 13/294,966 is a continuation of application No. PCT/US2011/060355, filed on Nov. 11, 2011.
(60) Provisional application No. 61/413,311, filed on Nov. 12, 2010.

(51) Int. Cl. C12Q 1/34

(52) U.S. Cl. CPC C12Q 1/34
USPC 435/5; 435/18

(57) ABSTRACT

A compound, or a pharmaceutically acceptable salt, ester, hydrate or solvate thereof, comprising formula I:

A-B
wherein A comprises a carbohydrate that is a neuraminidase or galactosidase substrate;
B comprises an odorant moiety;
B is covalently bonded to an anomeric carbon of A; and
A is enzymatically cleavable from B at the covalent bond site between A and B.
   Also disclosed are methods for detecting a pathogen with neuraminidase activity or galactosidase activity that include contacting the compound with a sample that may include the pathogen.

### CROSS REFERENCE TO RELATED APPLICATIONS

This application is a divisional of U.S. application Ser. No. 13/294,966, filed Nov. 11, 2011, which claims the benefit of U.S. Provisional Application No. 61/413,311, filed Nov. 12, 2010, and which claims priority to International Application No. PCT/US2011/060355, filed Nov. 11, 2011. All of the above-listed applications are herein incorporated by reference in their entirety.

### ACKNOWLEDGMENT OF GOVERNMENT SUPPORT

This invention was made with government support under Contract No. DE-AC52-06NA25396 between the United States Department of Energy and Los Alamos National Security, LLC for the operation of Los Alamos National Laboratory. The U.S. Government has certain rights in the invention.

### FIELD

This disclosure concerns odorant compounds and their use for the detection of pathogens, such as pathogens with neuraminidase activity or galactosidase activity.

### BACKGROUND

Human respiratory viruses, such as influenza A, B and C, respiratory syncytial virus and human rhinovirus accounted for an estimated 100 million infections in 2000 (Hughes and LeDuc, CDC Morbidity and Mortality weekly report 49(RR-3):1-54, 2000) in the US and the CDC reports that respiratory illnesses were the 5^{th} leading cause of death in the US in 2002 (Bridges et al., CDC Morbidity and Mortality weekly report 51(RR-3):Jan. 31, 2002). Although novel antiviral drugs for the treatment of influenza, such as neuraminidase inhibitors, became available recently, their efficiency depends on an early diagnosis (within the first two days of infection). The avian flu strain H5N1 currently does not transmit directly between people, but of the just over 100 documented human infections between 1997 and 2005 reported by the World Health Organization, the mortality rate was 54 percent. Current planning for the emergence of a human to human transmissible form of avian flu suggest the likely outbreak to start with the traditional pattern of index case appearance in the Asian region followed by rapid spread to Europe and to the continental US. Containment plans and measures proposed by US health agencies range from reducing public exposure risk to closing all US borders for any travel and most trade until a vaccine is developed and distributed. However, the development and validation of a vaccine can take as long as six months and the economic and human impact of quarantine, isolation and restricted flow of goods and people would clearly impact not only the US, but the world, on an unprecedented scale. A high-throughput, instrument-fee diagnostic measure to permit early warning and screening for infection, for instance at the borders and “in the field,” would mitigate the impact of countermeasures to an avian influenza outbreak.

### SUMMARY

According to one embodiment disclosed herein, there is provided a compound, or a pharmaceutically acceptable salt, ester, hydrate or solvate thereof, comprising formula I:

A-B

wherein A comprises a carbohydrate that is a neuraminidase or galactosidase substrate;

B comprises an odorant moiety;

B is covalently bonded to an anomeric carbon of A; and

A is enzymatically cleavable from B at the covalent bond site between A and B.

According to a further embodiment disclosed herein, there is provided a compound, or a pharmaceutically acceptable salt, ester, hydrate or solvate thereof, of formula II:

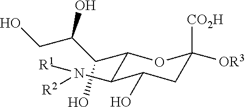

wherein

R¹ and R² are each individually selected from H, a carbonyl-containing group, lower alkyl, and glycol; and

R³ is an odorant moiety.

According to another embodiment disclosed herein, there is provided a compound, or a pharmaceutically acceptable salt, ester, hydrate or solvate thereof, of formula III:

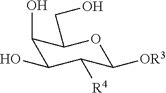

wherein:

R³ is an odorant moiety; and

R⁴ is a hydroxyl or —NHC(O)CH₃.

According to another embodiment disclosed herein, there is provided a compound, or a pharmaceutically acceptable salt, ester, hydrate or solvate thereof, of formula IV:

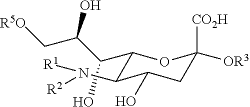

wherein

R¹ and R² are each individually selected from H, a carbonyl-containing group, lower alkyl, and glycol;

R³ is an odorant moiety; and

R⁵ is a moiety that includes at least one reactive functional group that has an affinity for at least one reactive functional group located on a solid surface.

Also disclosed herein is a composition or an article of manufacture that includes at least one of the compounds of formula I, II, III or IV.

A further disclosure herein is directed to a method for making a compound comprising:

reacting an anomeric carbon of a neuraminic acid residue or a galactose residue with a reactive oxygen-containing functional group of an odorant molecule to produce a neuraminic acid-odorant compound, or a galactose-odorant compound, respectively.

Also disclosed herein is a method for detecting the presence in a subject of a respiratory pathogen with neuraminidase activity or galactosidase activity, comprising:

(i) administering at least one compound of formula I, II, III or IV to the nasal passage of the subject; and

(ii) detecting the presence or absence of an odor by smell, wherein the presence of the odor results from release of the odorant moiety and indicates the presence of the respiratory pathogen.

In another embodiment, the method for detecting the presence of a respiratory pathogen with neuraminidase activity or galactosidase activity in a subject comprises:

(i) obtaining a sample (such as mucus or lavage fluid sample) from the respiratory tract of the subject;

(ii) contacting the sample with at least one compound of formula I, II, III or IV; and

(iii) detecting the presence or absence of an odor by smell, wherein the presence of the odor results from release of the odorant moiety and indicates the presence of the respiratory pathogen.

A further embodiment disclosed herein is directed to a method of detecting a pathogen with neuraminidase activity or galactosidase activity on a solid surface, wherein at least one compound of formula I, II, III or IV is disposed on the solid surface; the method comprising detecting the presence or absence of an odor by smell, wherein the presence of the odor results from release of the odorant moiety and detects the pathogen on the solid surface.

Another embodiment disclosed herein is directed to a method for detecting the presence of Vibrio cholerae in a sample, comprising:

(i) contacting at least one compound of formula I, II, III or IV with the sample; and

(ii) detecting the presence or absence of an odor by smell, wherein the presence of the odor results from release of the odorant moiety and detects Vibrio cholerae in the sample.

Also disclosed herein is a method for detecting a neuraminidase or a galactosidase in a sample, comprising:

(i) contacting at least one compound of formula I, II, III or IV with the sample; and

(ii) detecting the presence or absence of an odor by smell, wherein the presence of the odor results from release of the odorant moiety and detects a neuraminidase or a galactosidase in a sample.

The foregoing and other features and advantages will become more apparent from the following detailed description, which proceeds with reference to the accompanying figures.

### BRIEF DESCRIPTION OF THE DRAWINGS

FIG. 1 shows an example of a compound disclosed herein and the odorant molecule release product of cleavage of the compound by neuraminidase.

FIG. 2 depicts an example of a synthetic scheme for making neuraminic acid compounds as disclosed herein and a list of illustrative odorant moieties.

FIG. 3 shows an example of a compound disclosed herein and the odorant molecule release product of cleavage by neuraminidase, as well as detection of the release odorant molecule.

FIG. 4 shows an example of a compound that did not undergo cleavage in the presence of neuraminidase, which indicates selectivity of the enzymatic process.

FIG. 5 shows several additional exemplary synthetic schemes for making compounds as disclosed herein.

FIG. 6 shows an example of a compound disclosed herein and the odorant molecule release product of cleavage of the compound by β-galactosidase.

FIG. 7 shows an example of a synthetic scheme for making galactose compounds disclosed herein and a list of illustrative odorant moieties.

FIG. 8A shows that the 9-O position of a compound disclosed herein can be tethered to a solid surface by addition of a moiety (a “tether”) that includes at least one reactive functional group (such as an acyl, alkyl or azido group) that has affinity for the solid surface. FIG. 8B depicts a synthetic route for generating a tethered neuraminic acid-odorant compound, wherein R′ represents the tether group.

### DETAILED DESCRIPTION

### I. Abbreviations

GlcNAc N-acetylglucosamine

HPIV human parainfluenza virus

NA neuraminidase

NeuAc neuraminic acid

NeuNAc N-acetylneuraminic acid

### II. Terms and Methods

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

Acyl: A group represented by the formula RC(O)— wherein R represents an alkyl, particularly a lower alkyl.

Administer: As used herein, administering a composition or compound to a subject means to give, apply or bring the composition into contact with the subject or a sample obtained from the subject. Administration can be accomplished by any of a number of routes, such as, for example, intranasal. As used herein, “self-administration” refers to administration of a compound to a subject in which the subject is primarily responsible for applying or bringing the compound into contact with the subject.

Alkyl: A branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. A “lower alkyl” group is a saturated branched or unbranched hydrocarbon having from 1 to 5 carbon atoms. Alkyl groups may be substituted alkyls wherein one or more hydrogen atoms are substituted with a substituent such as halogen, cycloalkyl, alkoxy, amino, hydroxyl, aryl, or carboxyl. For example, an “alkoxyalkyl” has the structure —ROR, wherein R is an alkyl group.

Amine or amino: A group of the formula —NRR′, where R and R′ can be, independently, hydrogen or an alkyl, alkenyl, alkynyl, aryl, aralkyl, cycloalkyl, halogenated alkyl, or heterocycloalkyl group.

Amide or amido: A groups represented by the formula —C(O)NRR′, where R and R′ independently can be a hydrogen, alkyl, alkenyl, alkynyl, aryl, aralkyl, cycloalkyl, halogenated alkyl, or heterocycloalkyl group.

β-galactosidase: A hydrolase enzyme that catalyzes the hydrolysis of β-galactosides into monosaccharides. Substrates of different β-galactosidases include ganglioside GM1, lactosylceramides, lactose, and various glycoproteins. As used herein, a pathogen with “β-galactosidase activity” is one that expresses a functional β-galactosidase enzyme.

Carbonyl: A carbonyl group is a functional group comprising a carbon atom connected to an oxygen atom via a double bond. Carbonyl-containing groups include any substituent containing a carbon-oxygen double bond (C═O), including acyl groups, amides, carboxy groups, esters, ureas, carbamates, carbonates and ketones and aldehydes, such as substituents based on —COR or —RCHO where R is an aliphatic, heteroaliphatic, alkyl, heteroalkyl, hydroxyl, or a secondary, tertiary, or quaternary amine.

Carboxyl moiety: Any moiety or group that includes —C(O)O—. Illustrative carboxyl moieties include carboxylic acid (—C(O)OH); a carboxylate ester (—C(O)OR wherein R is an aliphatic or heteroaliphatic group); a carboxylate salt (—C(O)OM) wherein M is a cation such as Li, Na or K.

Cellulosic Substrate: Materials comprising, at least in part, cellulose. Cellulosic substrates include, but are not limited to, cotton, linen, rayon, wood, paper, cardboard, cellophane, etc.

Covalent bond: An interatomic bond between two atoms, characterized by the sharing of one or more pairs of electrons by the atoms. The terms “covalently bound” or “covalently linked” refer to making two separate molecules into one contiguous molecule.

Derivative: A compound or portion of a compound that is derived from or is theoretically derivable from a parent compound.

Galactosidase: An enzyme that catalyzes the conversion of galactosides to monosaccharides (such as galactose). There are two forms of galactosidase: α-galactosidase and β-galactosidase.

Haemophilus influenzae: A non-motile Gram-negative rod-shaped bacterium that causes a wide range of clinical illnesses. Some strains of Haemophilus influenzae are encapsulated, while other strains are unencapsulated. Encapsulated strains are classified on the basis of their distinct capsular antigens. There are six generally recognized types of encapsulated H. influenzae: a, b, c, d, e, and f. Genetic diversity among unencapsulated strains is greater than within the encapsulated group. Unencapsulated strains are termed nontypable (NTHi) because they lack capsular serotypes, however they can be classified by multi-locus sequence typing. The pathogenesis of H. influenzae infections is not completely understood, although the presence of the capsule in encapsulated type b (Hib), a serotype causing conditions such as epiglottitis, is known to be a major factor in virulence. Their capsule allows Haemophilus influenzae to resist phagocytosis and complement-mediated lysis in the non-immune host. The unencapsulated strains are almost always less invasive, however they can produce an inflammatory response in humans which can lead to many symptoms. Vaccination with Hib conjugate vaccine is effective in preventing Hib infection. Several vaccines are now available for routine use against Hib, however vaccines are not yet available against NTHi. Most strains of H. influenzae are opportunistic pathogens. They usually live in their host without causing disease, but cause problems only when other factors (such as a viral infection or reduced immune function) create an opportunity. H. influenzae expresses neuraminidase that can cleave α2,3-linked sialic acids.

Hydroxyl: A group represented by the formula —OH.

Hydroxyalkyl: An alkyl group that has at least one hydrogen atom substituted with a hydroxyl group. The term “alkoxyalkyl group” is defined as an alkyl group that has at least one hydrogen atom substituted with an alkoxy group described above.

Influenza virus: A segmented negative-strand RNA virus that belongs to the Orthomyxoviridae family. There are three types of Influenza viruses, A, B and C. Influenza A viruses infect a wide variety of birds and mammals, including humans, horses, marine mammals, pigs, ferrets, and chickens. In animals, most influenza A viruses cause mild localized infections of the respiratory and intestinal tract. However, highly pathogenic influenza A strains, such as H5N1, cause systemic infections in poultry in which mortality may reach 100%. H5N1 is also referred to as “avian influenza.” Influenza A viruses can be further classified into subtypes based on allelic variations in antigenic regions of two genes that encode surface glycoproteins, namely, hemagglutinin (HA) and neuraminidase (NA) which are required for viral attachment and cellular release. The host range of influenza B virus is significantly more limited, with only human, seals and ferrets known to be susceptible to influenza B. Influenza C virus infects humans, dogs and pigs and generally causes mild illness.

In influenza A viruses, nine different NA subtypes have been identified (N1, N2, N3, N4, N5, N6, N7, N8 and N9). Influenza NA is involved in the destruction of the cellular receptor for the viral HA by cleaving terminal neuraminic acid (also called sialic acid) residues from carbohydrate moieties on the surfaces of infected cells. NA also cleaves sialic acid residues from viral proteins, preventing aggregation of viruses. Using this mechanism, it is hypothesized that NA facilitates release of viral progeny by preventing newly formed viral particles from accumulating along the cell membrane, as well as by promoting transportation of the virus through the mucus present on the mucosal surface. Different influenza virus neuraminidases have varying specificities for sialic acid linkages. For example, avian influenza preferentially recognizes α2,3-linked sialic acids over α2,6-linked sialic acids, which are the target of most human strains (Glaser et al., J. Virol. 79(17):11533-11536, 2005).

Neuraminidase (NA): A glycoside hydrolase enzyme that cleaves the glycosidic linkages of neuraminic acids (also called sialic acids). Neuraminidases are also known as sialidases. Neuraminidase enzymes are a large family of enzymes expressed by a range of organisms, including viruses and bacteria. The most commonly studied neuraminidase is influenza virus neuraminidase.

Neuraminidase activity: As used herein, a pathogen with “neuraminidase activity” refers to a pathogen that encodes and expresses neuraminidase. Pathogens that express neuraminidase include viral pathogens (such as influenza viruses and parainfluenza viruses) and bacterial pathogens (for example, Haemophilus influenzae, Streptococcus pneumonia and Pseudomonas aeruginosa).

Odorant: A substance (e.g., a chemical moiety or molecule) that is detectable by a human or non-human animal olfactory perception and/or by suitable detection instruments. The odorant may be detectable by smell and/or it may cause olfactory irritation. The olfactory detection, as well as amplification by multiple substrate turnover, allows high sensitivity of the detection event. If applied directly to the mucous membrane of the nose, presymptomatic detection of the pathogen presence becomes feasible.

Parainfluenza virus: An enveloped, single-stranded negative sense RNA virus of the Paramyxoviridae family. Parainfluenza viruses express hemagglutinin-neuraminidase glycoprotein spikes on their surface. The parainfluenza virus hemagglutinin-neuraminidase protein has both hemagglutinating and neuraminidase activity within a single protein. There are four serotypes of human parainfluenza virus (HPIV-1 to -4). HPIVs can cause repeated infections throughout life, usually manifested by an upper respiratory tract illness (e.g., a cold and/or sore throat). HPIVs can also cause serious lower respiratory tract disease with repeat infection (e.g., pneumonia, bronchitis, and bronchiolitis), especially among the elderly, and among patients with compromised immune systems. Each of the four HPIVs has different clinical and epidemiologic features. The most distinctive clinical feature of HPIV-1 and HPIV-2 is croup (i.e., laryngotracheobronchitis). Both HPIV-1 and -2 can cause other upper and lower respiratory tract illnesses. HPIV-3 is more often associated with bronchiolitis and pneumonia. HPIV-4 is infrequently detected, possibly because it is less likely to cause severe disease. The incubation period for HPIVs is generally from 1 to 7 days. HPIVs are spread from respiratory secretions through close contact with infected persons or contact with contaminated surfaces or objects.

HPIV neuraminidases recognize α2,3-linked and α2,6-linked sialic acids (Suzuki et al., J. Virol. 75(10):4604-4613, 2001; Zhang et al., J. Virol. 79(2):1113-1124, 2005). Previous studies have demonstrated that HPIV-1 and HPIV-3 preferentially bind neolacto-series gangliosides containing a terminal N-acetylneuraminic acid (NeuAc) linked to N-acetyllactosamine (Galβ1-4GlcNAc) by the α2,3 linkage (NeuAcα2-3Galβ1-4GlcNAc). HPIV-3 is also capable of binding gangliosides with a terminal NeuAc linked to Galβ1-4GlcNAc through an α2-6 linkage (NeuAcα2-6Galβ1-4GlcNAc) or to gangliosides with an N-glycolylneuraminic acid (NeuGc) linked to Galβ1-4GlcNAc (NeuGcα2-3Galβ1-4GlcNac).

Pathogen: A biological agent that causes disease or illness to its host. Pathogens include, for example, bacteria, viruses, fungi, protozoa and parasites. Pathogens are also referred to as infectious agents. In some embodiments of the methods disclosed herein, the pathogen is a respiratory pathogen, such as an influenza virus. In other embodiments, the pathogen is a bacterial pathogen, such as Haemophilus influenzae, Streptococcus pneumoniae, Pseudomonas aeruginosa or Vibrio cholerae. In particular embodiments herein, the pathogen is one that encodes a neuraminidase protein. In other embodiments, the pathogen is one that encodes β-galactosidase.

Pharmaceutically acceptable salt or ester: Salts or esters prepared by conventional means that include basic salts of inorganic and organic acids, including but not limited to hydrochloric acid, hydrobromic acid, sulfuric acid, phosphoric acid, methanesulfonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, mandelic acid and the like. “Pharmaceutically acceptable salts” of the presently disclosed compounds also include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)aminomethane, and tetramethylammonium hydroxide. These salts may be prepared by standard procedures, for example by reacting the free acid with a suitable organic or inorganic base. Any chemical compound recited in this specification may alternatively be administered as a pharmaceutically acceptable salt thereof. “Pharmaceutically acceptable salts” are also inclusive of the free acid, base, and zwitterionic forms. Descriptions of suitable pharmaceutically acceptable salts can be found in Handbook of Pharmaceutical Salts, Properties, Selection and Use, Wiley VCH (2002). When compounds disclosed herein include an acidic function such as a carboxy group, then suitable pharmaceutically acceptable cation pairs for the carboxy group are well known to those skilled in the art and include alkaline, alkaline earth, ammonium, quaternary ammonium cations and the like. Such salts are known to those of skill in the art. For additional examples of “pharmacologically acceptable salts,” see Berge et al., J. Pharm. Sci. 66:1 (1977). “Pharmaceutically acceptable esters” includes those derived from compounds described herein that are modified to include a hydroxy or a carboxyl group. An in vivo hydrolysable ester is an ester, which is hydrolysed in the human or animal body to produce the parent acid or alcohol. Suitable pharmaceutically acceptable esters for carboxy include C₁₋₆ alkoxymethyl esters for example methoxy-methyl, C₁₋₆ alkanoyloxymethyl esters for example pivaloyloxymethyl, phthalidyl esters, C₃₋₈ cycloalkoxycarbonyloxyC₁₋₆ alkyl esters for example 1-cyclohexylcarbonyl-oxyethyl; 1,3-dioxolen-2-onylmethyl esters for example 5-methyl-1,3-dioxolen-2-onylmethyl; and C₁₋₆ alkoxycarbonyloxyethyl esters for example 1-methoxycarbonyl-oxyethyl which may be formed at any carboxy group in the compounds.

An in vivo hydrolysable ester containing a hydroxy group includes inorganic esters such as phosphate esters and α-acyloxyalkyl ethers and related compounds which as a result of the in vivo hydrolysis of the ester breakdown to give the parent hydroxy group. Examples of α-acyloxyalkyl ethers include acetoxy-methoxy and 2,2-dimethylpropionyloxy-methoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. Examples of substituents on benzoyl include morpholino and piperazino linked from a ring nitrogen atom via a methylene group to the 3- or 4-position of the benzoyl ring.

For therapeutic use, salts of the compounds are those wherein the counter-ion is pharmaceutically acceptable. However, salts of acids and bases which are non-pharmaceutically acceptable may also find use, for example, in the preparation or purification of a pharmaceutically acceptable compound.

The pharmaceutically acceptable acid and base addition salts as mentioned hereinabove are meant to comprise the therapeutically active non-toxic acid and base addition salt forms which the compounds are able to form. The pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic (i.e. hydroxybutanedioic acid), tartaric, citric, methanesulfonic, ethanesulfonic, benzenesulfonic, p-toluenesulfonic, cyclamic, salicylic, p-aminosalicylic, pamoic and the like acids. Conversely said salt forms can be converted by treatment with an appropriate base into the free base form.

The compounds containing an acidic proton may also be converted into their non-toxic metal or amine addition salt forms by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, salts with organic bases, e.g. the benzathine, N-methyl-D-glucamine, hydrabamine salts, and salts with amino acids such as, for example, arginine, lysine and the like.

The term “addition salt” as used hereinabove also comprises the solvates which the compounds described herein are able to form. Such solvates are for example hydrates, alcoholates and the like.

Protected derivatives of the disclosed compounds also are contemplated. The term “protecting group” or “blocking group” refers to any group that when bound to a functional group prevents or diminishes the group's susceptibility to reaction. “Protecting group” generally refers to groups well known in the art which are used to prevent selected reactive groups, such as carboxy, amino, hydroxy, mercapto and the like, from undergoing undesired reactions, such as nucleophilic, electrophilic, oxidation, reduction and the like. The terms “deprotecting,” “deprotected,” or “deprotect,” as used herein, are meant to refer to the process of removing a protecting group from a compound.

Pseudomonas aeruginosa: A Gram-negative, aerobic, rod-shaped bacterium. P. aeruginosa is an opportunist pathogenic that causes diseases in animals, including humans. It is found in soil, water, skin flora and most man-made environments. The symptoms of P. aeruginosa infections are generalized inflammation and sepsis. If colonization occurs in critical body organs, such as the lungs, the urinary tract, and kidneys, the results can be fatal. Because P. aeruginosa thrives on most surfaces, this bacterium is also found on and in medical equipment, including catheters, causing cross-infections in hospitals and clinics. P. aeruginosa is known to bind the GalNAcβ1,4Gal moiety on asialylated glycolipids and expresses a type of neuraminidase that can cleave α2,3-linked sialic acids (Soong et al., J. Clin. Invest. 116(8):2297-2305, 2006).

Respiratory pathogen: Refers to a type of pathogen that infects cells of the respiratory system.

Sample: Refers to any biological or environmental sample. In some embodiments, the sample is a biological sample obtained from a subject, such as a mucous, saliva, blood, urine or fecal sample. In other embodiments, the sample is an environmental sample, such as a liquid sample (for example, water or sewage) or a soil sample.

Shigella dysenteriae: A species of the Gram-negative, rod-shaped bacterial genus Shigella. Shigella can cause shigellosis (bacillary dysentery). S. dysenteriae, spread by contaminated water and food, causes the most severe dysentery because of its potent and deadly Shiga toxin, but other species may also be dysentery agents. Contamination is often caused by bacteria on unwashed hands during food preparation, or soiled hands reaching the mouth.

Solid surface: In the context of the present disclosure, a “solid surface” refers to any non-liquid or non-gas surface upon which a compound of the present disclosure can be applied (for example, applied in a suitable solution). In some embodiments, the solid surface is an item that can be used to obtain a sample from a subject (such as a mucous sample), for example a cellulosic substrate such as paper, a swab, tissue, test strip or wipe. In other embodiments, the solid surface is an object that can trap or adhere to pathogens present in aerosols, such as an air filter, a respiratory mask or an item of clothing. In other embodiments, the solid surface is any surface within a room or building, such as a public building (e.g., airport, medical office, school etc.). In particular examples, the solid surface is a floor, a counter, a wall, a piece of furniture, or a piece of laboratory or medical equipment. In other embodiments, the solid surface is a surface on a subject, such as the skin. In some embodiments, the compounds of the present disclosure are modified to allow for attachment (e.g., via a covalent bound between the compound and the solid surface) to a solid surface, such as for attachment to a cellulosic substrate such as paper, a swab, a tissue, a test strip or a wipe.

Streptococcus pneumoniae: A Gram-positive, alpha-hemolytic anaerobe that is a significant human pathogen and a major cause of pneumonia. S. pneumoniae also causes acute sinusitis, otitis media, meningitis, bacteremia, sepsis, osteomyelitis, septic arthritis, endocarditis, peritonitis, pericarditis, cellulitis and brain abscess. S. pneumoniae is the most common cause of bacterial meningitis in adults, children, and dogs, and is one of the primarily isolates found in ear infections. S. pneumoniae is part of the normal upper respiratory tract flora, but can become pathogenic under the right conditions (for example, immune suppression). S. pneumoniae expresses a type of neuraminidase that can cleave α2,3-linked sialic acids. S. pneumoniae also expresses β-galactosidase and thus can be detected using the galactose-odorant compounds disclosed herein.

Subject: Living multi-cellular vertebrate organisms, a category that includes both human and non-human mammals, such as non-human primates. In some embodiments herein, the subject is a human.

Vibrio cholerae: A Gram-negative comma-shaped bacterium with a polar flagellum. The unencapsulated serogroup V. cholerae O1 and encapsulated V. cholerae O139 cause epidemic and pandemic outbreaks of cholera. Cholera is an acute bacterial infection of the intestine caused by ingestion of food or water contaminated with O1 or O139 V. cholerae. V. cholerae expresses β-galactosidase and neuraminidase and thus can be detected using the galactose-odorant or the neuraminic acid-odorant compounds disclosed herein.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### III. Introduction

Current technologies for the detection of pathogens are biased towards the human preference for optical readout of results. Disclosed herein is a radically different approach that employs the most discriminatory human sense—smell—coupled with an odorant releasing and pathogen-specific catalyzed event. This approach provides an instrumentation-free, high-throughput, field-able, and inexpensive assay directly on one of the most ubiquitous sensors—the nose.

Odorants for food, pharmaceutical and cosmetic applications span a wide structure space of molecules with several hundred known and available compounds. The discriminatory power of smell receptors to distinguish structural features is utilized in methods disclosed herein as an enzymatic release of a “free” odorant will be clearly distinguishable from any signal a substrate odorant chimera might produce. In addition to “pleasant” olfactory stimulation and for instance for uncooperative patients or critical emergency detection needs, the release of irritants (e.g., capsaicins which are the irritants from peppers) can be incorporated into the compounds. The wide range of possible odorants further allows multiplexed detection of multiple pathogens triggering different olfactory responses.

### IV. Compounds

In one embodiment disclosed herein there are provided compounds, or a pharmaceutically acceptable salt, ester, hydrate or solvate thereof, represented by the formula I:

A-B

wherein

A comprises a carbohydrate that is a neuraminidase or galactosidase substrate;

B comprises an odorant moiety;

B is covalently bonded to an anomeric carbon of A; and

A is enzymatically cleavable from B at the covalent bond site between A and B.

The A carbohydrate may be a neuraminic acid residue or a galactose residue. The neuraminic acid residue may be selected, for example, from neuraminic acid or an N-substituted neuraminic acid. The galactose reside may be selected, for example, from galactose or N-acetylgalactosamine. The A carbohydrate includes an anomeric center and purified α-anomers, purified β-anomers, or α, β mixtures may be used.

The B odorant may be any chemical structure that can be covalently bonded to the A carbohydrate and is susceptible to enzymatic cleavage from A via a neuraminidase or galactosidase. The B odorant moiety is derived from odorant molecules that include at least one oxygen-containing functional group that is reactive with the anomeric carbon of A. Such molecules typically include at least one ester, aldehyde, ketone, amine, thiol and/or hydroxyl functional group that is reactive with the anomeric carbon of A. The reaction between the O-containing functional group of the B odorant and the anomeric carbon of the A carbohydrate results in the formation of an —O— covalent linkage between A and B. Illustrative odorant moieties include those derived from zingerone, folrosia, vanillin, javanol, methyl diantilis, nonadienol, citronellol, mefresol, anisyl alcohol, cyclohexyl propanol, dihydroeugenol, cinnamyl alcohol, floral pyranol, peony alcohol, geraniol, ionone, ebanol, sandalore, citronellal, benzyl acetone, celery acetone, cetone, claritone, isomuscone, damascone delta, dimethyl octenone, ethyl amyl ketone, exaltone, exaltenone, geranyl acetone, globanone, hedione, jasmatone, jasmone cis, methyl napthyl ketone, methyl undecyl ketone, nerone, plicatone, velvione and vetikone.

The A carbohydrate typically is covalently bonded to the B odorant via an —O— linkage (i.e., structure of A-O-B), but can include other linkages through N or S. Specific enzymatic cleavage at the —O— linkage releases the odorant moiety.

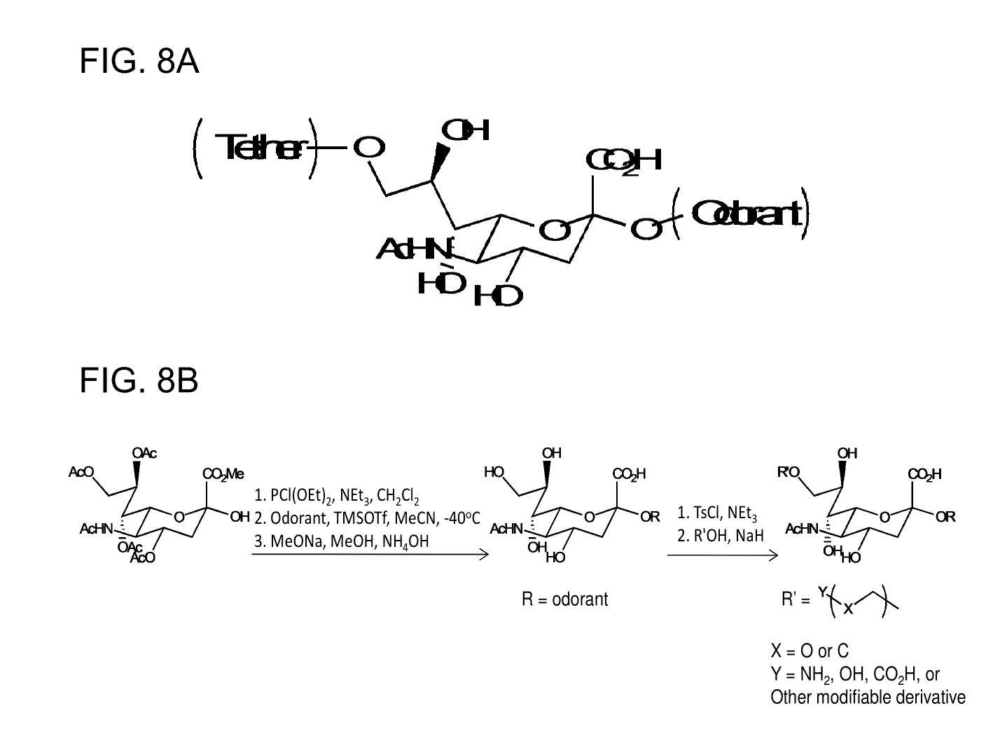

Certain embodiments of the compounds of formula I may also include a reactive functional moiety for coupling (e.g., via covalently bonding) the compounds to a solid surface as described in more detail below and in FIGS. 8A and 8B.

Structural variants of the compounds can be designed to provide certain characteristics as desired. One characteristic may be for enzyme specificity. Another characteristic may be for cleavage rate.

In another embodiment disclosed herein there are provided compounds represented by formula II:

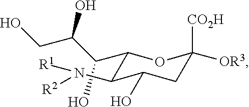

or a pharmaceutically acceptable salt, ester, hydrate or solvate thereof,

wherein:

R¹ and R² are each individually selected from H, a carbonyl-containing group, lower alkyl, and glycol; and

R³ is an odorant moiety.

In certain embodiments, R¹ and R² are each individually selected from H, acyl, or lower alkyl. Particularly preferred are H, acetyl, formyl, methyl, ethyl, and propyl. In more specific embodiments, R¹ is H and R² is acetyl, formyl, or propyl.

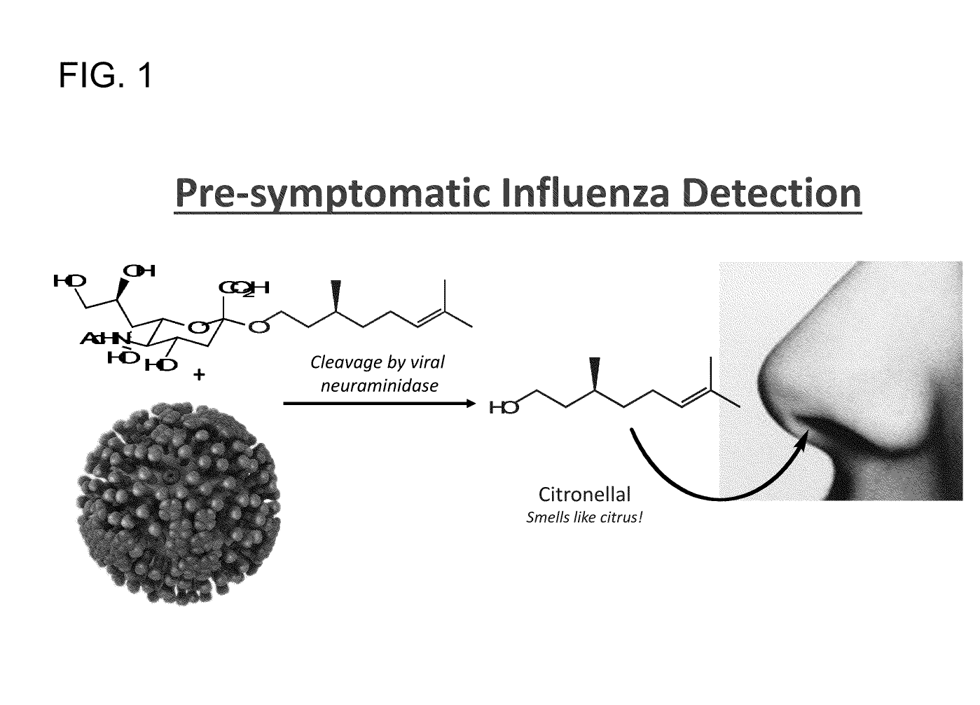

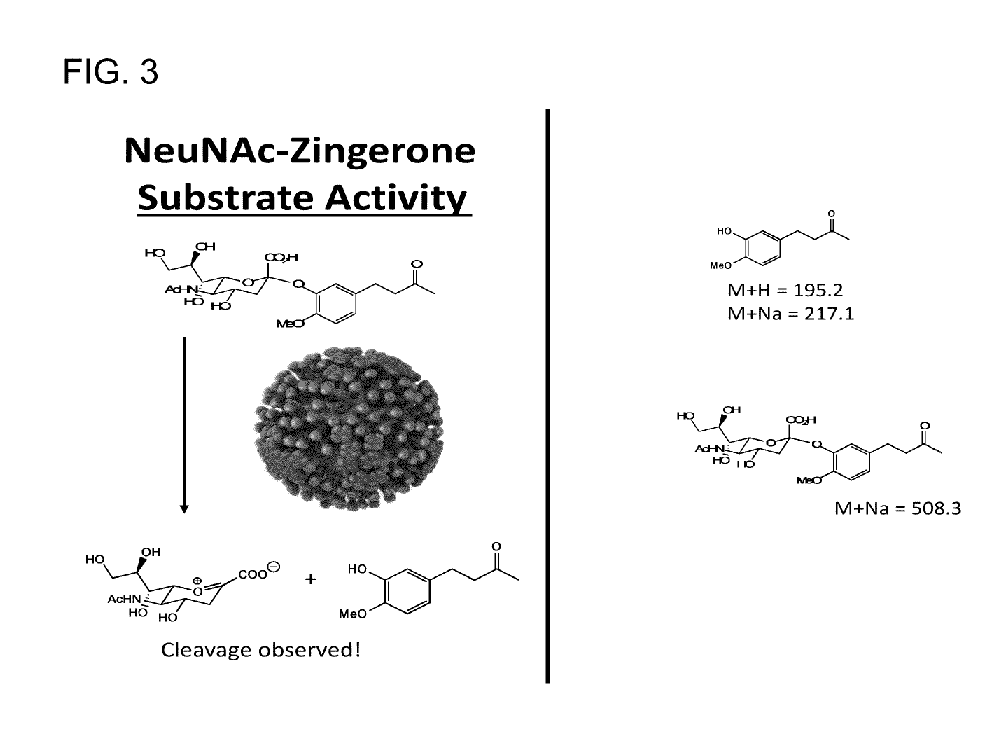

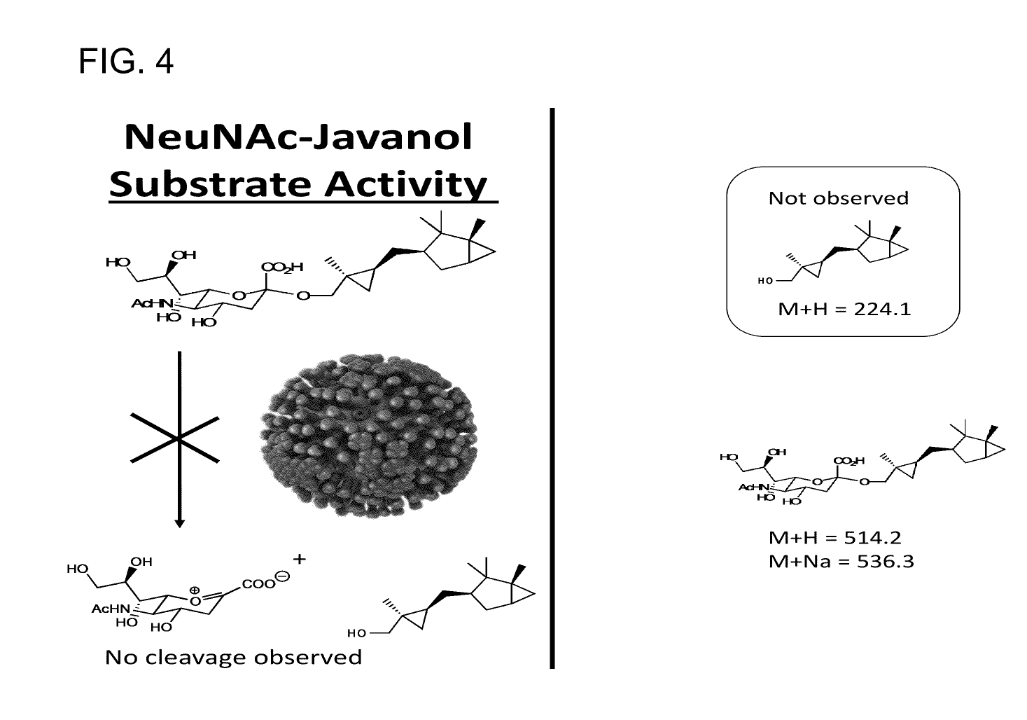

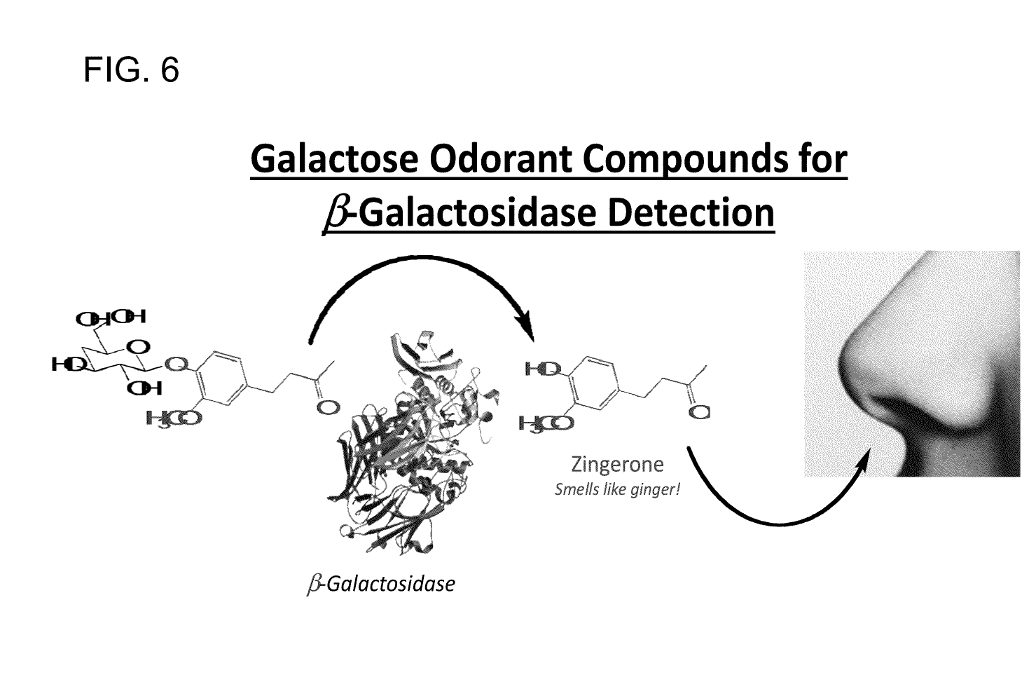

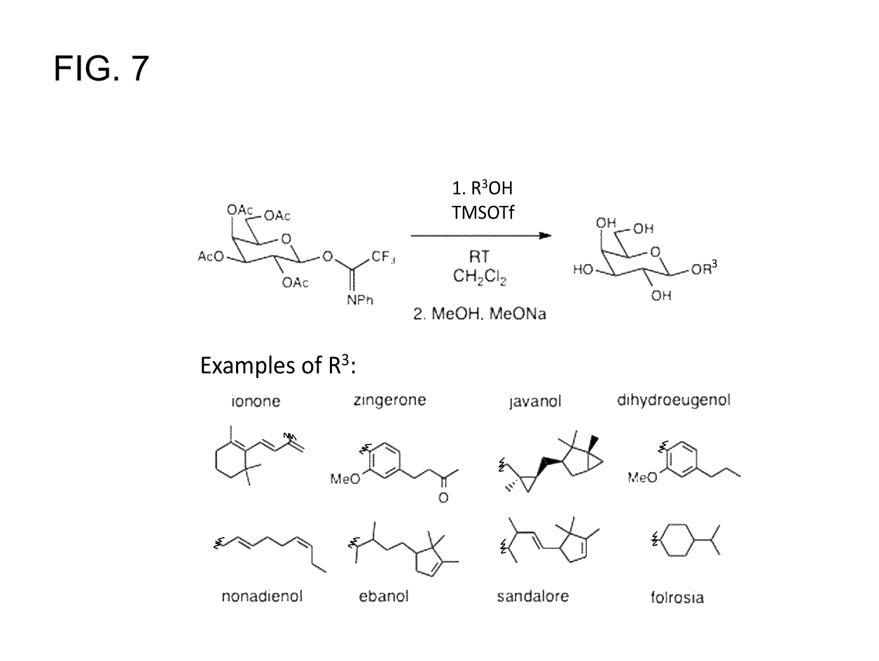

Illustrative R³ odorant moieties are described above and are shown in FIGS. 1-7. The R³ odorant moiety is covalently bonded to an anomeric carbon of the ring structure of formula II. The anomeric carbon in the structure of formula II is located at the 2-carbon position.

Certain embodiments of the compounds of formula II may also include a reactive functional moiety for coupling (e.g., via covalently bonding) the compounds to a solid surface as described in more detail below and in FIGS. 8A and 8B.

In a further embodiment disclosed herein there are provided compounds represented by the formula III:

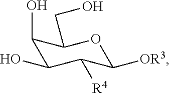

or a pharmaceutically acceptable salt, ester, hydrate or solvate thereof,

wherein R³ is an odorant moiety as described above; and

R⁴ is a hydroxyl or —NHC(O)CH₃.

The R³ odorant moiety is covalently bonded to an anomeric carbon of the ring structure of formula III. The anomeric carbon in the structure of formula III is located at the 1-carbon position.

Certain embodiments of the compounds of formula III may also include a reactive functional moiety for coupling (e.g., via covalently bonding) of the compounds to a solid surface as described in more detail below and in FIGS. 8A and 8B.

A further embodiment disclosed herein is directed to modifying the compounds disclosed herein to include at least one moiety that includes at least one reactive functional group that has an affinity for at least one reactive functional group located on a solid surface such as a cellulosic substrate. For example, disclosed herein are compounds, or a pharmaceutically acceptable salt, ester, hydrate or solvate thereof, represented by the formula IV:

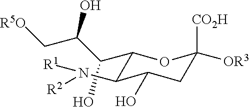

wherein:

R¹ and R² are each individually selected from H, a carbonyl-containing group, lower alkyl, and glycol;

R³ is an odorant moiety; and

R⁵ is a moiety that includes at least one reactive functional group that has an affinity for at least one reactive functional group located on a solid surface.

In certain embodiments, R¹ and R² are each individually selected from H, acyl, or lower alkyl. Particularly preferred are H, acetyl, formyl, methyl, ethyl, and propyl. In more specific embodiments, R¹ is H and R² is acetyl, formyl, or propyl.

Illustrative R³ odorant moieties are described above and are shown in FIGS. 1-7. The R³ odorant moiety is covalently bonded to an anomeric carbon of the ring structure of formula IV. The anomeric carbon in the structure of formula IV is located at the 2-carbon position.

Illustrative R⁵ moieties include at least one reactive functional group that can form a covalent bond with a reactive functional group on a solid surface such as a cellulosic substrate. Illustrative R⁵ reactive groups for forming the covalent bond include acyl, alkyl, azido, amino, amido, hydroxy, a carboxyl-containing moiety, thiol, aldehyde, epoxy, sulfonamide, and halogen. In certain examples, R⁵ may also include a linker group such as an aliphatic chain (e.g., an alkyl chain derived from a fatty acid or a polyalkylene polymer or oligomer), or a polyalkylene oxide (e.g., polyethylene glycol) that links the compound scaffold disclosed herein to the covalently-reactive functional group. An example of a compound of formula IV is shown in FIG. 8B wherein R′ (designated R⁵ in formula IV) includes a linker group —(—X—CH₂—)— and a reactive functional group (Y).

In certain embodiments, the solid surface may inherently include reactive functional groups that can form covalent bonds with the R⁵ functional group. In other embodiments, the solid surface may be chemically modified to introduce reactive functional groups so that such functional groups can covalently bond with the compounds disclosed herein. For instance, a cellulosic substrate can be oxidized to create aldehyde functional groups that can directly react with an amino group of the compounds disclosed herein (e.g., an amino group that is included in R⁵ in the compound of formula IV). In other embodiments, an amino or carboxyl reactive group in R⁵ may be reacted to form a peptide bond with a carboxyl or amino reactive group, respectively, on the solid surface.

### V. Synthesis Methods

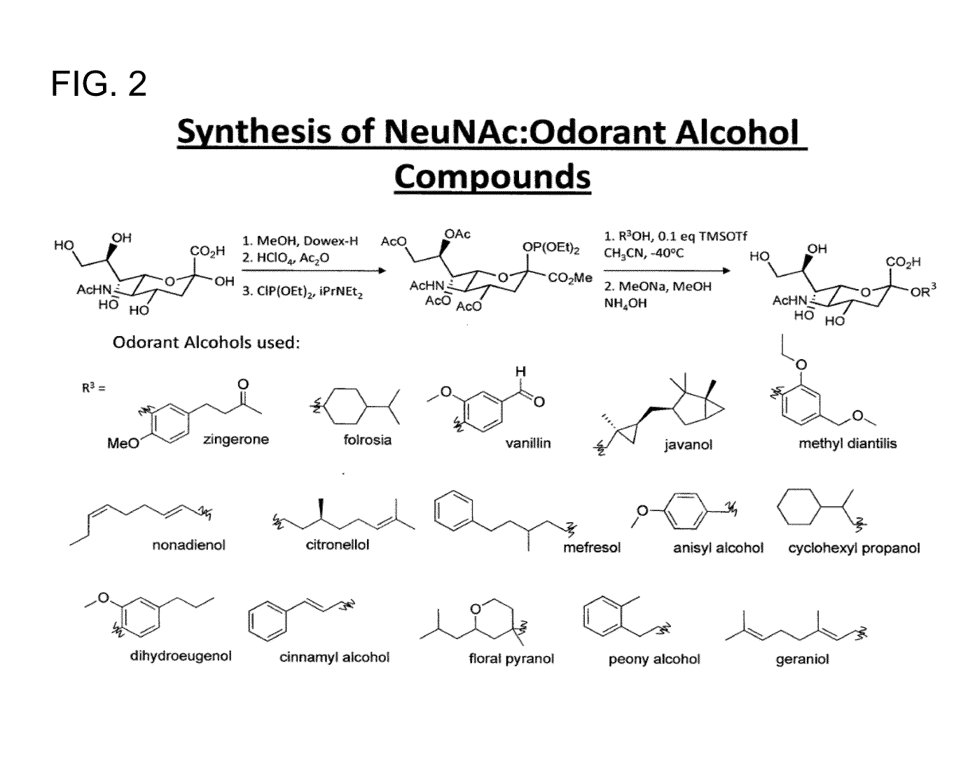

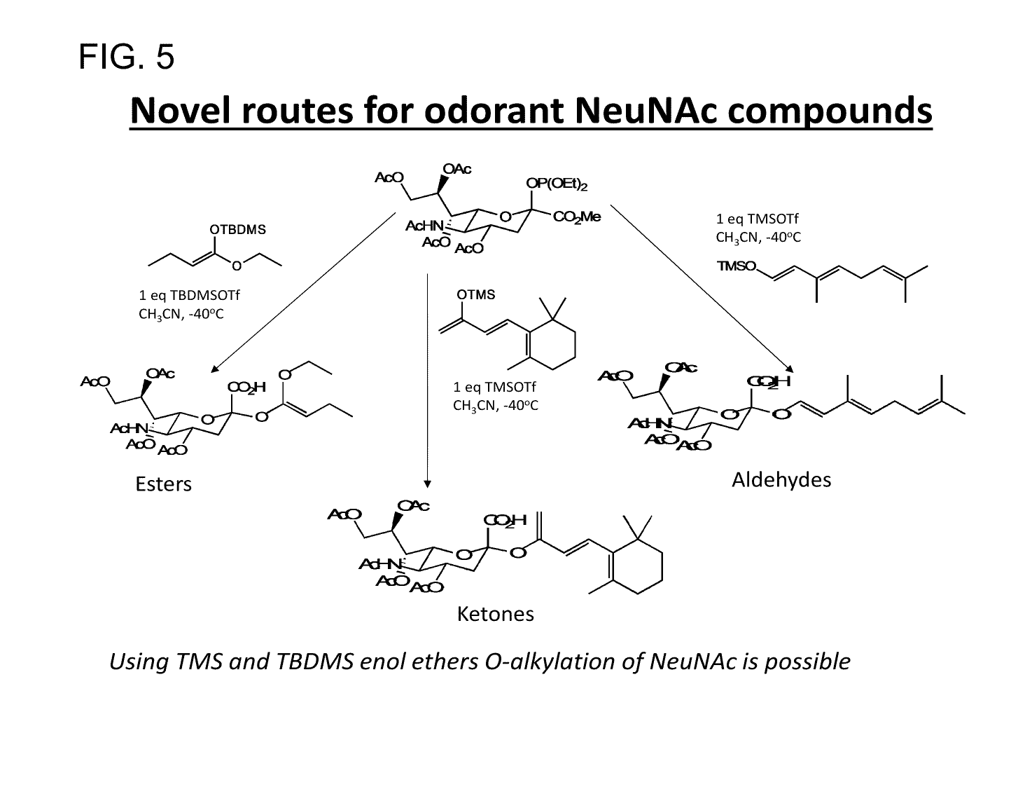

The compounds disclosed herein may be generally synthesized by the methods described below and as shown in FIGS. 2, 5,7 and 8B. The neuraminic acid residue or galactose residue is protected by acetylation and methylester formation. Selective removal of the anomeric acetylester, and activation as cyanocarbamate allows the silyltriflate mediated condensation with the odorant hydroxyl. The silylating reagent may be, for example, trimethylsilyl (TMS), trimethylsilyl trifluoromethane sulfonate (TMSOTf), t-butyldimethylsilyl trifluoromethane sulfonate (TBDMSOTf), triisopropylsilyl trifluoromethanesulfonate (TIPSOTf) or tert-butyldiphenylsilyl trifluoromethylsulfonate (TBDPSOTf). A final deprotection with sodium methoxide yields the final product.

### VI. Compositions

Another aspect of the present disclosure relates to compositions that include the above-described compounds of formula I, II, III or IV. The compositions may be for direct administration to a subject or for application to a solid surface as described below in more detail in section VII. The compositions include a detectable amount of the compounds of formula I, II, III or IV. The compositions may include only one compound of formula I, II, III or IV, or the compositions may include a plurality of the compounds of formula I, II, III or IV.

The compositions can include at least one further additive (preferably a pharmaceutically acceptable additive) such as carriers, thickeners, diluents, buffers, preservatives, surface active agents and the like in addition to the active compound of formula I, II, III or IV.

In general, the nature of the carrier will depend on the particular mode of application being employed. For instance, fluid formulations (e.g., nasal spray) usually contain pharmaceutically and physiologically acceptable fluids such as water, physiological saline, balanced salt solutions, aqueous dextrose, glycerol or the like as a vehicle. For solid compositions, conventional non-toxic solid carriers can include, for example, pharmaceutical grades of mannitol, lactose, starch, or magnesium stearate. In addition to biologically-neutral carriers, the compositions can contain minor amounts of non-toxic auxiliary substances, such as wetting or emulsifying agents, preservatives, and pH buffering agents and the like, for example sodium acetate or sorbitan monolaurate.

Compositions disclosed herein include those formed from pharmaceutically acceptable salts and/or solvates of the disclosed compounds. Pharmaceutically acceptable salts include those derived from pharmaceutically acceptable inorganic or organic bases and acids. Particular disclosed compounds possess at least one basic group that can form acid—base salts with acids. Examples of basic groups include, but are not limited to, amino and imino groups. Examples of inorganic acids that can form salts with such basic groups include, but are not limited to, mineral acids such as hydrochloric acid, hydrobromic acid, sulfuric acid or phosphoric acid. Basic groups also can form salts with organic carboxylic acids, sulfonic acids, sulfo acids or phospho acids or N-substituted sulfamic acid, for example acetic acid, propionic acid, glycolic acid, succinic acid, maleic acid, hydroxymaleic acid, methylmaleic acid, fumaric acid, malic acid, tartaric acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid, and, in addition, with amino acids, for example with α-amino acids, and also with methanesulfonic acid, ethanesulfonic acid, 2-hydroxymethanesulfonic acid, ethane-1,2-disulfonic acid, benzenedisulfonic acid, 4-methylbenzenesulfonic acid, naphthalene-2-sulfonic acid, 2- or 3-phosphoglycerate, glucose-6-phosphate or N-cyclohexylsulfamic acid (with formation of the cyclamates) or with other acidic organic compounds, such as ascorbic acid. In particular, suitable salts include those derived from alkali metals such as potassium and sodium, alkaline earth metals such as calcium and magnesium, among numerous other acids well known in the pharmaceutical art.

Certain compounds include at least one acidic group that can form an acid—base salt with an inorganic or organic base. Examples of salts formed from inorganic bases include salts of the presently disclosed compounds with alkali metals such as potassium and sodium, alkaline earth metals, including calcium and magnesium and the like. Similarly, salts of acidic compounds with an organic base, such as an amine (as used herein terms that refer to amines should be understood to include their conjugate acids unless the context clearly indicates that the free amine is intended) are contemplated, including salts formed with basic amino acids, aliphatic amines, heterocyclic amines, aromatic amines, pyridines, guanidines and amidines. Of the aliphatic amines, the acyclic aliphatic amines, and cyclic and acyclic di- and tri-alkyl amines are particularly suitable for use in the disclosed compounds. In addition, quaternary ammonium counterions also can be used.

Particular examples of suitable amine bases (and their corresponding ammonium ions) for use in the present compounds include, without limitation, pyridine, N,N-dimethylaminopyridine, diazabicyclononane, diazabicycloundecene, N-methyl-N-ethylamine, diethylamine, triethylamine, diisopropylethylamine, mono-, bis- or tris-(2-hydroxyethyl)amine, 2-hydroxy-tert-butylamine, tris(hydroxymethyl)methylamine, N,N-dimethyl-N-(2-hydroxyethyl)amine, tri-(2-hydroxyethyl)amine and N-methyl-D-glucamine. For additional examples of “pharmacologically acceptable salts,” see Berge et al., J. Pharm. Sci. 66:1 (1977).

Compounds disclosed herein can be crystallized and can be provided in a single crystalline form or as a combination of different crystal polymorphs. As such, the compounds can be provided in one or more physical form, such as different crystal forms, crystalline, liquid crystalline or non-crystalline (amorphous) forms. Such different physical forms of the compounds can be prepared using, for example different solvents or different mixtures of solvents for recrystallization. Alternatively or additionally, different polymorphs can be prepared, for example, by performing recrystallizations at different temperatures and/or by altering cooling rates during recrystallization. The presence of polymorphs can be determined by X-ray crystallography, or in some cases by another spectroscopic technique, such as solid phase NMR spectroscopy, IR spectroscopy, or by differential scanning calorimetry.

To formulate the compositions, the compound can be combined with various additives, as well as a base or vehicle for dispersion of the compound. Desired additives include, but are not limited to, pH control agents, such as arginine, sodium hydroxide, glycine, hydrochloric acid, citric acid, and the like. In addition, local anesthetics (for example, benzyl alcohol), isotonizing agents (for example, sodium chloride, mannitol, sorbitol), adsorption inhibitors (for example, Tween 80 or Miglyol 812), solubility enhancing agents (for example, cyclodextrins and derivatives thereof), stabilizers (for example, serum albumin), and reducing agents (for example, glutathione) can be included. Adjuvants, such as aluminum hydroxide (for example, Amphogel, Wyeth Laboratories, Madison, N.J.), Freund's adjuvant, MPL™ (3-O-deacylated monophosphoryl lipid A; Corixa, Hamilton, Ind.) and IL-12 (Genetics Institute, Cambridge, Mass.), among many other suitable adjuvants well known in the art, can be included in the compositions.

The compound can be dispersed in a base or vehicle, which can include a hydrophilic compound having a capacity to disperse the compound, and any desired additives. The base can be selected from a wide range of suitable compounds, including but not limited to, copolymers of polycarboxylic acids or salts thereof, carboxylic anhydrides (for example, maleic anhydride) with other monomers (for example, methyl (meth)acrylate, acrylic acid and the like), hydrophilic vinyl polymers, such as polyvinyl acetate, polyvinyl alcohol, polyvinylpyrrolidone, cellulose derivatives, such as hydroxymethylcellulose, hydroxypropylcellulose and the like, and natural polymers, such as chitosan, collagen, sodium alginate, gelatin, hyaluronic acid, and nontoxic metal salts thereof. Often, a biodegradable polymer is selected as a base or vehicle, for example, polylactic acid, poly(lactic acid-glycolic acid) copolymer, polyhydroxybutyric acid, poly(hydroxybutyric acid-glycolic acid) copolymer and mixtures thereof. Alternatively or additionally, synthetic fatty acid esters such as polyglycerin fatty acid esters, sucrose fatty acid esters and the like can be employed as vehicles. Hydrophilic polymers and other vehicles can be used alone or in combination, and enhanced structural integrity can be imparted to the vehicle by partial crystallization, ionic bonding, cross-linking and the like. The vehicle can be provided in a variety of forms, including fluid or viscous solutions, gels, pastes, powders, microspheres and films for direct application to a solid surface.

The compositions can also be formulated as a solution, microemulsion, or other ordered structure suitable for high concentration of active ingredients. The vehicle can be a solvent or dispersion medium containing, for example, water, ethanol, polyol (for example, glycerol, propylene glycol, liquid polyethylene glycol, and the like), and suitable mixtures thereof. Proper fluidity for solutions can be maintained, for example, by the use of a coating such as lecithin, by the maintenance of a desired particle size in the case of dispersible formulations, and by the use of surfactants. In many cases, it will be desirable to include isotonic agents, for example, sugars, polyalcohols, such as mannitol and sorbitol, or sodium chloride in the composition.

The compositions of the present disclosure typically are sterile and stable under conditions of manufacture, storage and use. Sterile solutions can be prepared by incorporating the compound in the required amount in an appropriate solvent with one or a combination of ingredients enumerated herein, as required, followed by filtered sterilization. Generally, dispersions are prepared by incorporating the compound and/or other biologically active agent into a sterile vehicle that contains a basic dispersion medium and the required other ingredients from those enumerated herein. In the case of sterile powders, methods of preparation include vacuum drying and freeze-drying which yields a powder of the compound plus any additional desired ingredient from a previously sterile-filtered solution thereof. The prevention of the action of microorganisms can be accomplished by various antibacterial and antifungal agents, for example, parabens, chlorobutanol, phenol, sorbic acid, thimerosal, and the like.

### VII. Instrument-Free Detection of Viral and Bacterial Pathogens

The common receptor motifs found on influenza A, B and C viruses and parainfluenza viruses are neuraminidase (NA) and hemagglutinin (HA). There are an estimated 50 copies of tetrameric NA and in excess of 100 copies of trimeric HA on the surface of an influenza viral particle (Murti and Webster, Virology 149:36-43, 1986) that recognize specific neuraminic acid (Neu) residues on the host cell for binding, aggregation and entry into the cell. As cornerstones of the infection process, the recognition elements for cell surface adhesion are highly conserved and specific for a viral strain.

The recognition molecules for influenza viruses A, B, C and avian influenza, as well as certain pathogenic respiratory bacteria, are N-acetylneuraminic acid residues located at the carbohydrate termini of various cell surface glycoproteins (Saito and Yu, “Biochemistry and function of sialidases” in Biology of the Sialic Acids, 1995, pages 261-313). In the case of influenza virus, to release newly synthesized mature influenza particles from an infected cell, influenza neuraminidase cleaves off the acetyl-neuraminic acid residues on the cellular receptors. The enzyme activity of various viral and bacterial pathogens is exploited in the methods disclosed herein to release an odorant from synthetic substrates. For example, human influenza A viruses are known to preferentially cleave α2,6-linked sialic acids, while avian influenza viruses are generally specific for α2,3-linked sialic acid moieties. Human parainfluenza viruses bind both α2,3-linked and α2,6-linked neuraminic acid residues. In addition, the bacterial respiratory pathogens Haemophilus influenzae, Streptococcus pneumonia and Pseudomonas aeruginosa recognize α2,3-linked sialic acids.

Upper respiratory viral and bacterial agents primarily infect the mucus membrane and surface cells in the nose, which is also a highly sensitive and discriminatory biosensor of olfactory receptors. The methods disclosed herein allow for the direct detection of respiratory infections, as well as for the detection of other types of pathogens that possess neuraminidase activity. In some embodiments, the methods allow for the detection of bacteria that express galactosidase (such as the detection of Streptococcus pneumonia to test for bacterial pneumonia in a subject, or the detection of Vibrio cholerae to test for contaminated water). Certain embodiments of the disclosed methods utilize the sense of smell and therefore require no external instrumentation. Smells are detected in the nose by specialized receptor cells of the olfactory epithelium situated on the olfactory receptor neurons (Luu et al., J Neurosci 24(45):10128-10137, 2004). Each neuron sends a nerve axon to the olfactory bulb, the brain structure just above the nose. The sensory perception of odorants is primarily derived from the interaction of multiple receptors along the nose with small organic molecules, although the sense of taste may also be involved. While taste is a fairly crude sense (there are only four values that your tongue can sense—sweet, salty, sour and bitter), the nose can sense and differentiate thousands of different odors by a panel of response from more that 300 receptors (Buck and Axel, Cell 65:175-187, 1991). The G-protein coupled cascades amplify signal transmission of odorant detection and can provide sensitivities for volatile compounds below the parts-per-billion (ppb) range. Common reported sensitivity limits are based on concentrations of the odorant in air. In some embodiments of the methods disclosed herein, the detection and sensitivity limits of the methods should improve by several orders of magnitude as the odorant is directly released within the mucous membrane of the nose.

In other embodiments of the disclosed method the odor may be detected by instruments such as a scentometer, olfactometer or electronic nose, or by a secondary observer, including humans and other mammals such as dogs, who provides the feedback after detection of the odorant.

A. Methods of Detecting Respiratory Pathogens Directly in the Nose

In some embodiments of the methods described herein, the substrate-odorant compounds of the present disclosure can be used to detect the presence of a respiratory pathogen with neuraminidase activity (i.e. a pathogen that expresses functional neuraminidase) or galactosidase activity (i.e. a pathogen that expresses a functional galactosidase, such as β-galactosidase) directly in the nose of a subject to be tested. Such a method is useful as a screening tool or system to identify individuals that are infected with a respiratory pathogen (such as a pandemic influenza virus) to prevent further spread of the infectious agent. For example, subjects can be tested before crossing a border or boarding an airplane, and the results of the test can be used to make decisions that might limit the spread of disease. The instrument-free detection methods disclosed herein combine several signal amplification steps that result in a highly sensitive assay: the presence of multiple NA copies on the surface of the pathogen to be detected; the catalytic substrate cleavage; the direct and unique pathogen-specific release of the trigger on the sensor and the biological signal cascade of smell; and the exquisite sensitivity of olfactory sensing. Due to the extreme sensitivity of the disclosed methods, the presence of a respiratory pathogen can be confirmed even prior to the subject developing symptoms.

The methods disclosed herein can detect any respiratory pathogen with neuraminidase activity, or any respiratory pathogen with galactosidase activity. The methods include administering a substrate-odorant compound described herein to the nasal passage(s) of a subject; and detecting the presence or absence of an odor (that is, the odor from the odorant moiety released by the substrate-odorant compound) by smell, wherein detection of the odor is performed by the subject, and wherein the presence of the odor indicates the presence of the respiratory pathogen. In some cases, the compound is self-administered by the subject. However, the compound can also be delivered by another individual, such as a health care provider. The compound can be administered using any suitable delivery device that administers an amount of the compound to the nasal passages of the subject sufficient to allow for detection of the respiratory pathogen. In particular examples, the compound is administered in a nasal spray or on a swab (e.g., a swab impregnated with the compound). The compound may also be administered to samples retrieved from the subject's respiratory tract, such as a mucus or lavage fluid sample.

In some embodiments, the respiratory pathogen with neuraminidase activity is a viral pathogen, such as an influenza virus or a parainfluenza virus. In other embodiments, the respiratory pathogen with neuraminidase activity is a bacterial pathogen, such as Haemophilus influenzae, Streptococcus pneumoniae or Pseudomonas aeruginosa. In some embodiments, the respiratory pathogen with galactosidase activity is a bacterial pathogen, such as Streptococcus pneumoniae.

B. Methods of Detecting Pathogens on Solid Surfaces or in Fluid Samples

Further disclosed herein are methods that allow for the detection of a neuraminidase-expressing, or a galactosidase-expressing, viral or bacterial pathogen on any type of solid surface, or in any type of fluid sample. The disclosed methods include applying a substrate-odorant compound as disclosed herein to the solid surface; and detecting the presence or absence of an odor by smell, wherein the presence of the odor detects the pathogen on the solid surface. The methods also encompass applying a substrate-odorant compound as disclosed herein to the fluid sample; and detecting the presence or absence of an odor by smell, wherein the presence of the odor detects the pathogen in the fluid sample.

The solid surface can be any type of non-liquid, non-gas surface that allows for the application of one or more of the compounds disclosed herein in an appropriate carrier or diluent. In some embodiments, the solid surface is an item that can be used to obtain a sample from a subject (such as a mucous sample), for example a cellulosic substrate such as paper, a swab, tissue, test strip or wipe. In other embodiments, the solid surface is an object that can trap or adhere to pathogens present in aerosols, such as an air filter, a respiratory mask or an item of clothing. In other embodiments, the solid surface is any surface within a room or building, such as a public building (e.g., airport, medical office, school etc.). In particular examples, the solid surface is a floor, a counter, a wall, a piece of furniture, or a piece of laboratory or medical equipment. In other embodiments, the solid surface is a surface on a subject, such as the skin. In some embodiments, the compound is modified to allow for attachment to a solid surface, such as for attachment to paper, a swab, a tissue, a test strip or a wipe.

The fluid sample can be any type of liquid sample, such as, for example, a gel, soap, hand-sanitizer or detergent. The fluid sample can also be a body fluid sample, such as urine, saliva, mucous, cerebrospinal fluid, blood or semen.

In some embodiments, the pathogen with neuraminidase activity is a viral pathogen, such as an influenza virus or a parainfluenza virus. In other embodiments, the pathogen with neuraminidase activity is a bacterial pathogen, such as Haemophilus influenzae, Streptococcus pneumonia, Pseudomonas aeruginosa, Vibrio cholerae or Shigella dysenteriae. In some embodiments, the pathogen with galactosidase activity is a bacterial pathogen, such as Streptococcus pneumonia or Vibrio cholerae.

The methods disclosed herein can be used for a wide variety of purposes, including for the detection of a pathogen with neuraminidase activity (or galactosidase activity) in or on a subject (such as in a mucous sample from the subject, or on the subject's skin), or on the surface of an object.

In particular examples, the methods can be used to detect the presence of a pathogen on a swab taken from a subject suspected of or at risk of being infected with the pathogen. Similarly, the disclosed methods can be used to evaluate how well an individual (such as a health care provider or day care worker) has washed their hands by detecting the presence or absence of a pathogen on a wipe from the subject's hands. This specific method could also be used to evaluate the effectiveness of hand sanitizer to eliminate a particular pathogen from the human skin—for instance, the substrate-odorant compound can be applied along with the sanitizer or after application of the sanitizer.

In other examples, the disclosed methods are utilized to identify pathogens with neuraminidase activity or galactosidase activity (such as β-galactosidase activity) in a laboratory or medical setting. For example, one could test various types of medical or laboratory equipment, or any surface within a particular room of interest, to identify the presence of a pathogen.

The disclosed methods can also be used by medical workers or others to identify potential viral or bacterial contaminants. For example, a medical worker (or other, such as another first responder) that is entering an outbreak area could wear a respiratory mask upon which the substrate-odorant molecule has been applied. If the pathogen is present in aerosols, then the odorant will be released and the medical worker (or other) is alerted to the presence of the pathogen in the immediate vicinity by the presence of the smell.

Moreover, the substrate-odorant compounds of the present disclosure can be applied to an air filter in a room to continuously monitor for the presence of a pathogen, particularly a respiratory pathogen capable of transmission in aerosols.

C. Methods of Detecting the Presence of Vibrio cholerae in a Biological or Environmental Sample

The waterborne pathogen Vibrio cholerae expresses a β-galactosidase enzyme. Thus, the methods disclosed herein can be harnessed to detect the presence of Vibrio cholerae in a sample. The method includes contacting a substrate-odorant compound of the present disclosure with the sample and detecting the presence or absence of an odor by smell, wherein the presence of the odor detects Vibrio cholerae in the sample. In some embodiments, the sample is a liquid sample, such as a water sample or sewage sample. For example, a water sample can be taken from a river, pond, lake or the like where there is a concern for potential contamination. In other embodiments, the sample is a fecal sample. For example, the fecal sample can be taken from an individual suspected of being infected with Vibrio cholerae.

The following examples are provided to illustrate certain particular features and/or embodiments. These examples should not be construed to limit the disclosure to the particular features or embodiments described.

### EXAMPLES

### Example 1

### Synthesis of Substrate: Odorant Compounds

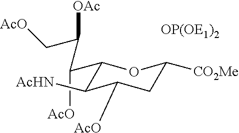

### 1-O-diethylphosphite-4,7,8,9-Tetra-O-acetyl-N-acetylneuraminic Acid Methyl Ester. (1)

4,7,8,9-Tetra-O-acetyl-N-acetylneuraminic Acid Methyl Ester (0.5 g, 1.0 mmol) was dissolved in dry AcCN (10 mL) with diisopropylethylamine (0.29 g, 2.2 mmol) under an Ar atmosphere at RT. Diethylchlorophosphite (0.31 g, 2.0 mmol) in 1 mL dry AcCN was added dropwise, and the reaction mixture was stirred overnight. The solvent was removed envacuo and the remaining oil was subject to flash chromatography, gradient of 10%-50% acetone:toluene. 30% acetone:toluene R_{f}=0.34. Ref: Schmidt, R. R. et al, Glycoconjugate Journal, 10, 16, 1993.

The following procedure was used for all alcohol sialic acid coupling reactions: Compound 1 (200 mg, 0.3 mmol) and odorant alcohol (3.0 mmol) were added to anhydrous acetonitrile (10 mL) in a flame dried flask. The reaction mixture was cooled to −40° C., and a solution of TMSOTf (16 mg, 0.06 mmol) in dry acetonitrile (0.25 mL) was added via syringe pump over 10 minutes. The solution was kept at −40° C. for 30 minutes then allowed to warm to 0° C., at which point 1 mL triethylamine was added to quench to lewis acid. The solvent was removed en vacuo and the oily remainder was subject to flash chromatography, gradient of 10%-50% acetone:toluene. The product was then taken up in methanol (2 mL) and NaOMe (2 mL 1M in MeOH) was added and let stir overnight. Ammonium hydroxide (2 mL) was then added further and the solution stirred for 2 hrs, after which DOWEX-H resin was added until the pH reached 6-7, and the solution was filtered. Solvent was removed en vacuo and purified by reverse phase flash chromatography using an acetonitrile:methanol gradient from 10-90% to yield the desired product. Overall yields varied from 20-50%.

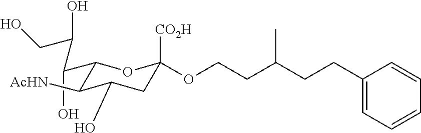 [SMILES: CC(=O)NC1C(O)CC(OCCC(C)CCc2ccccc2)(C(=O)O)OC1C(O)C(O)CO]

### Sodium (3-methyl-5-phenylpentanyl-5-acetamido-3,5-dideoxy-α-D-glycero-D-galacto-2-nonulopyranoside)onate

Peracetylated methyl ester: ¹H NMR (300 MHz, CDCl₃) δ 7.22 (m, 5H), 5.35 (m, 2H), 5.14 (m, 2H), 4.82 (m, 1H), 4.30 (m, 1H), 4.03 (m, 3H), 3.74 (m, 3H), 2.57 (m, 4H), 1.85-2.15 (m, 17H), 1.66 (m, 3H), 1.41 (m, 2H), 0.85 (m, 3H) ppm. LRMS m/z M+H 652.10 g/mol, M+Na 674.31 g/mol. Deacetylated carboxylate: ¹H NMR (300 MHz, d₆DMSO, D₂O) δ 7.19 (m, 5H), 3.23-3.78 (m, 8H), 2.61 (m, 2H), 2.21 (m, 1H), 1.90 (m, 5H), 1.81 (s, 3H), 1.68 (s, 6H), 1.21-1.60 (m, 6H), 0.86 (m, 3H) ppm LRMS m/z M+Na 492.22 g/mol

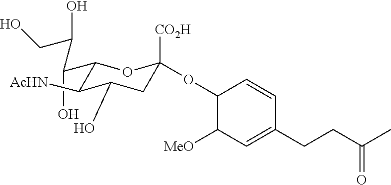 [SMILES: COC1C=C(CCC(C)=O)C=CC1OC1(C(=O)O)CC(O)C(NC(C)=O)C(C(O)C(O)CO)O1]

### Sodium (4-(4-hydroxy-3-methoxyphenyl)butan-2-one-5-acetamido-3,5-dideoxy-α-D-glycero-D-galacto-2-nonulopyranoside)onate

Peracetylated methyl ester: ¹H NMR (300 MHz, CDCl₃) δ 6.75 (d, 1H), 6.60 (m, 1H), 6.53 (m, 1H), 5.41 (m, 1H), 5.28 (m, 2H), 5.14 (m, 2H), 4.76 (m, 1H), 4.55 (m, 1H), 4.04 (m, 3H), 3.80 (s, 3H), 3.66 (s, 3H), 2.71 (m, 5H), 1.85-2.15 (m, 19H) ppm. LRMS m/z M+H 669.12 g/mol, M+Na 691.23 g/mol. Deacetylated carboxylate: ¹H NMR (300 MHz, d₆DMSO, D₂O) δ 6.70 (q, 1H), 6.58 (d, 1H), 6.51 (m, 1H). 3.63 (s, 3H), 3.19-3.60 (m, 7H), 2.62 (m, 5H), 2.00 (s, 3H), 1.85 (m, 4H) ppm. LRMS m/z M+Na 508.19 g/mol

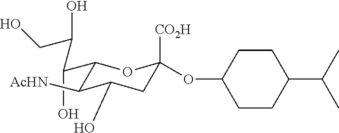 [SMILES: CC(=O)NC1C(O)CC(OC2CCC(C(C)C)CC2)(C(=O)O)OC1C(O)C(O)CO]

### Sodium (4-isopropylcyclohexyl-5-acetamido-3,5-dideoxy-α-D-glycero-D-galacto-2-nonulopyranoside)onate

Peracetylated methyl ester: ¹H NMR (300 MHz, CDCl₃) δ 5.30 (m, 1H), 5.12 (m, 2H), 4.95 (m, 1H), 4.78 (m, 1H), 4.31 (m, 1H), 4.06 (m, 3H), 3.74 (m, 3H), 2.52 (m, 2H), 1.85-2.15 (m, 18H), 1.41 (m, 4H), 0.95 (m, 2H), 0.85 (m, 6H) ppm. LRMS m/z M+H 616.04 g/mol, M+Na 638.28 g/mol. Deacetylated carboxylate: ¹H NMR (300 MHz, d₆DMSO, D₂O) δ 3.14-3.73 (m, 8H), 2.55 (m, 1H), 1.90 (m, 6H), 1.56 (m, 4H), 1.10 (m, 4H), 0.85 (m, 6H) ppm LRMS m/z M+Na 455.24 g/mol

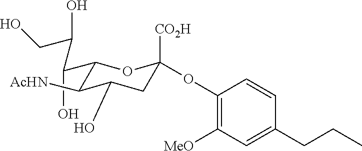 [SMILES: CCCc1ccc(OC2(C(=O)O)CC(O)C(NC(C)=O)C(C(O)C(O)CO)O2)c(OC)c1]

### Sodium (2-methoxy-4-n-propylphenyl-5-acetamido-3,5-dideoxy-α-D-glycero-D-galacto-2-nonulopyranoside)onate

Peracetylated methyl ester: ¹H NMR (300 MHz, CDCl₃) δ 6.60 (m, 3H), 5.46 (m, 3H), 5.12 (m, 1H), 4.95 (m, 1H), 4.78 (m, 1H), 4.31 (m, 1H), 4.10 (m, 3H), 3.80 (m, 3H), 3.60 (m, 3H), 2.52 (m, 2H), 1.85-2.15 (m, 17H), 1.50 (m, 2H), 0.81 (m, 3H) ppm. LRMS m/z M+Na 662.27 g/mol. Deacetylated carboxylate: LRMS m/z M+Na 479.21 g/mol

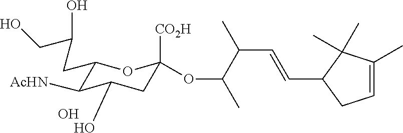

### Sodium ((Z)-3-methyl-5-(2,2,3-trimethyl-1-cyclopent-3-enyl)pent-4-enyl-5-acetamido-3,5-dideoxy-α-D-glycero-D-galacto-2-nonulopyranoside)onate

Peracetylated methyl ester: ¹H NMR (300 MHz, CDCl₃) δ 5.30 (m, 2H), 5.12 (m, 2H), 4.78 (m, 1H), 4.31 (m, 2H), 4.06 (m, 3H), 3.74 (m, 6H), 2.28 (m, 2H), 1.85-2.15 (m, 15H), 1.58 (m, 2H), 0.92 (m, 6H), 0.70 (m, 6H) ppm. LRMS m/z M+H 682.17 g/mol, M+Na 704.37 g/mol. Deacetylated carboxylate: ¹H NMR (300 MHz, d₆DMSO, D₂O) δ 5.39 (m, 3H), 3.89 (d, 1H), 3.14-3.73 (m, 7H), 2.60 (m, 2H), 2.21 (m, 1H), 1.90 (s, 3H), 1.81 (s, 3H), 1.21-1.45 (m, 4H), 0.90-1.10 (m, 6H), 0.72 (m, 3H) ppm LRMS m/z M+Na 521.30 g/mol

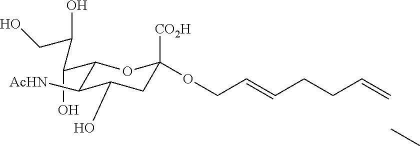

### Sodium ((E,Z)-2,6-nonadien-1-yl-5-acetamido-3,5-dideoxy-α-D-glycero-D-galacto-2-nonulopyranoside)onate

Peracetylated methyl ester: ¹H NMR (300 MHz, CDCl₃) δ 5.68 (m, 1H), 5.47 (m, 1H), 5.30 (m, 4H), 5.12 (m, 2H), 4.78 (m, 1H), 4.21 (m, 1H), 4.06 (m, 3H), 3.87 (m, 2H), 3.74 (s, 3H, CO₂CH₃), 2.52 (m, 1H), 1.85-2.15 (m, 21H), 1.48 (m, 2H), 1.25 (m, 3H) ppm. LRMS m/z M+H 613.97 g/mol, M+Na 636.24. Deacetylated carboxylate: ¹H NMR (300 MHz, d₆DMSO, D₂O) δ 5.25-5.60 (m, 4H), 3.25-3.75 (m, 9H), 2.60 (m, 1H), 1.85-2.07 (m, 7H), 0.87 (t, 3H) ppm LRMS m/z M+Na 453.23 g/mol

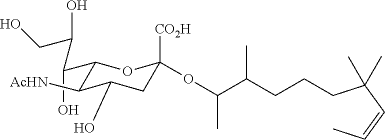 [SMILES: C/C=C\C(C)(C)CCCC(C)C(C)OC1(C(=O)O)CC(O)C(NC(C)=O)C(C(O)C(O)CO)O1]

### Sodium (3-methyl-5-(2,2,3-trimethyl-1-cyclopent-3-enyl)pentan-2-yl-5-acetamido-3,5-dideoxy-α-D-glycero-D-galacto-2-nonulopyranoside)onate

Peracetylated methyl ester: ¹H NMR (300 MHz, CDCl₃) δ 5.45 (m, 2H), 5.32 (m, 2H), 5.13 (m, 2H), 4.57 (m, 1H), 4.31 (m, 3H), 4.00 (m, 3H), 3.80 (m, 6H), 2.28 (m, 2H), 1.85-2.15 (m, 15H), 0.92 (m, 6H) ppm. LRMS m/z M+H 684.178 g/mol, M+Na 706.36 g/mol. Deacetylated carboxylate: ¹H NMR (300 MHz, d₆DMSO, D₂O) δ 5.40 (m, 1H), 3.89 (d, 1H), 3.14-3.73 (m, 8H), 2.60 (m, 2H), 2.21 (m, 1H), 1.90 (s, 3H), 1.81 (s, 3H), 1.68 (s, 6H), 1.21-1.45 (m, 6H), 0.90-1.10 (m, 5H), 0.72 (m, 4H) ppm LRMS m/z M+Na 523.29 g/mol

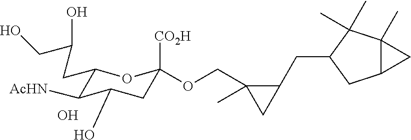

### Sodium ([(1R,2S)-1-methyl-2-[[(1R,3S,5S)-1,2,2-trimethyl-3-bicyclo[3.1.0]hexanyl]methyl]cyclopropyl]methyl-5-acetamido-3,5-dideoxy-α-D-glycero-D-galacto-2-nonulopyranoside)onate

Peracetylated methyl ester: ¹H NMR (300 MHz, CDCl₃) δ 6.09 (d, 1H), 5.45 (m, 3H), 5.32 (m, 2H), 4.88 (m, 1H), 4.38 (m, 1H), 4.19 (m, 2H), 3.80 (s, 3H), 2.61 (m, 1H), 1.85-2.15 (m, 19H), 1.06-1.30 (m, 10H), 0.92 (s, 3H), 0.81 (s, 3H), 0.50 (m, 3H), 0.01 (m, 2H) ppm. LRMS m/z M+H 684.178 g/mol, M+Na 706.36 g/mol. Deacetylated carboxylate: ¹H NMR (300 MHz, d₆DMSO, D₂O) δ 3.20-3.56 (m, 8H), 2.99 (dd, 1H), 2.62 (m, 2H), 1.89 (s, 3H), 1.85 (m, 1H), 1.26 (m, 3H), 0.7-0.98 (m, 15H), 0.42 (m, 2H), −0.15 (m, 2H) ppm. LRMS m/z M+Na 523.29 g/mol

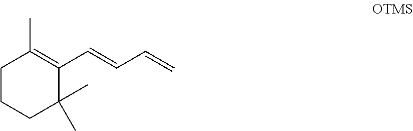

B-Ionone (0.5 g, 2.5 mmol) was added to anhydrous THF (20 mL) and cooled to −75° C. under an Ar balloon. KHMDS (0.55 g, 2.75 mmol) in anhydrous THF (10 mL) was added via syringe pump over 15 minutes, and let stir for 30 minutes. TMSCl (0.29 g, 2.75 mmol) was added dropwise and the reaction mixture was allowed to warm to 0° C. Solvent was removed envacuo yielding an oily liquid that was distilled under vacuum to yield pure product.

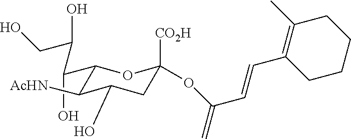 [SMILES: C=C(/C=C/C1=C(C)CCCC1)OC1(C(=O)O)CC(O)C(NC(C)=O)C(C(O)C(O)CO)O1]

### Sodium ((E)-4-(2,6,6-trimethyl-1-cyclohexenyl)but-1-3-dienyl-5-acetamido-3,5-dideoxy-α-D-glycero-D-galacto-2-nonulopyranoside)onate

Compound 1 (200 mg, 300 mmol) and TMS enol ether of Ionone (800 mg, 3.0 mol) were added to anhydrous AcCN (10 mL) in a flame dried flask. The reaction mixture was cooled to −40° C., and a solution of TMSOTf (80 mg, 300 mmol) in AcCN (0.8 mL) was added via syringe pump over 10 minutes. The solution was kept at −40° C. for 30 minutes then allowed to warm to 0° C., at which point 1 mL triethylamine was added to quench to reaction. The solvent was removed envacuo and the oily remainder was subject to flash chromatography, gradient of 10%-50% acetone:toluene. Peracetylated methyl ester: ¹H NMR (300 MHz, CDCl₃) δ 6.59 (d, 1H), 5.71 (d, 1H), 5.36 (m, 3H), 5.25 (m, 2H), 5.09 (m, 1H), 4.62 (m, 1H), 4.31 (d, 1H), 4.15 (m, 4H), 3.98 (m, 1H), 3.75 (s, 3H), 2.58 (m, 1H), 1.85-2.15 (m, 15H), 1.60 (2H), 1.43 (m, 2H), 1.01 (m, 6H) ppm. ¹³C NMR (75 MHz, CDCl₃) δ 171.03, 170.87, 170.66, 170.46, 170.28, 167.33, 153.82, 136.68, 131.19, 129.22, 128.41, 127.24, 98.80, 92.30, 72.26, 71.83, 68.91, 68.02, 62.23, 53.14, 49.13, 39.88, 38.79, 34.33, 33.34, 31.14, 29.07, 29.05, 28.97, 23.39, 21.79, 21.35, 21.21 ppm. LRMS m/z M+Na 688.21 g/mol.

### Example 2

### Enzymatic Cleavage Resulting in Odorant Release

### Influenza Culture

Reference strains A/Bejing/262/95 (H1N1), A/Sydney/04/97 (H3N2), and B/Harbin/07/94 were obtained from Tricore Laboratories (Albuquerque, N. Mex.). Novel swine-origin H1N1 A/California/07/2009 was provided by the Centers for Disease Control (Atlanta, Ga.). Viruses were cultured in MDCK cells (ATCC, Manassas, Va.) using high glucose DMEM without phenol red (Invitrogen, Carlsbad, Calif.). Viruses were cultured until cytopathic effect was observed in the host cells (4-7 days). Samples were clarified using low speed centrifugation followed by filtration through a 0.2 μm membrane. Virus samples were stored with 0.5% BSA fraction V (Invitrogen) at 4° C. until use. Fresh samples were used within two weeks after harvest to reduce degradation of virus.

### Competitive Inhibition Assay

To determine the effectiveness of odorant binding to viral neuraminidase, the NA-Star™ assay (Applied Biosystems, Foster City) was used with slight modifications to kit directions. Briefly, the virus was serially diluted two-fold in NA-Star™ assay buffer across a white optical 96-well plate and warmed to 35° C. NA-Star™ substrate was added to the viral dilutions and the samples were incubated 10 minutes at 35° C. Accelerant was added and the luminescence was read with a 0.5 second integration time on a Biotek plate reader. The dilution of virus that resulted in a 40:1 signal to noise ratio was used for the competitive inhibition assay. Odorants were diluted in half-log dilutions from 1.6 nM to 160 μM. Odorant plate and diluted virus were warmed to 35° C. before addition of virus to odorants. NA-Star™ substrate was added and the samples were incubated and read as described previously.

### Viral Neuraminidase Purification

Viral neuraminidase was purified from a large scale culture of influenza. A minimum of 2 L of culture was concentrated using a 30,000 MW Amicon filter, followed by incubation with 1 mg/ml TPCK-treated trypsin (Worthington Biochemical Corp., Lakewood, N.J.) at 37° C. for one hour. The reaction was stopped by the addition of 0.85 mg/ml soybean trypsin inhibitor (Worthington Biochemical Corp.). Viral cores were pelleted for one hour at 30,000 RPM using a Ti-SW32 rotor and Optima L-90K ultracentrifuge (Beckman Coulter, Brea, Calif.). The supernatant was transferred to a fresh tube and viral neuraminidase was pelleted for 60 h at 30,000 RPM. Neuraminidase was resuspended in 100 mM ammonium acetate, pH 5.5.

### Viral Cleavage of Odorant

Viral samples were concentrated 10-fold using a 30,000 MW Amicon filter (Millipore, Billerica, Mass.). After concentration, viral media was exchanged for 100 mM ammonium acetate buffer, pH 5.5. Concentration of neuraminidase in the viral samples was analyzed using the NA-Star™ kit as described previously. Samples were diluted to achieve a signal to noise ratio of 80:1 for the viral cleavage assay. Odorants were added to the virus preparation for a final concentration of 400 μg/ml. Virus and odorant were incubated overnight at 35° C. followed by addition of methanol and centrifugal filtration through a 10,000 MW Ultracel membrane (Millipore). Filtrate was analyzed by FTQ ESI mass spectrometry.

### Assay Results

As a crude mixture, decrease of the substrate signal and appearance of the cleavage products signal indicated whether the substrate was cleaved and odorant released. Results using odorant zingerone show a significant loss of peak at 508.3 amu, which relates to the substrate (M+Na), and the appearance of zingerone peaks at 195.2 (M+H), 217.1 (M+Na), and a peak at 292.1 (M+H) representing the cleaved neuraminic acid. Results using odorant javanol show a peak at 514.2 (M+H) and 536.3 (M+Na) amu, suggesting the substrate is still present. These results indicate potential selectivity and varying rates with different odorant structures.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A method for detecting the presence of a respiratory pathogen with neuraminidase activity or galactosidase activity in a subject, comprising:
(i) administering to the nasal passage of the subject at least one compound, or a pharmaceutically acceptable salt, ester, hydrate or solvate thereof, comprising the formula A-B, wherein A comprises a carbohydrate that is a neuraminidase or galactosidase substrate, B comprises an odorant moiety, B is covalently bonded to an anomeric carbon of A, and A is enzymatically cleavable from B at the covalent bond site between A and B; and
(ii) detecting the presence or absence of an odor by smell, wherein the presence of the odor results from release of the odorant moiety and indicates the presence of the respiratory pathogen.

2. The method of claim 1, wherein A comprises a neuraminic acid residue or a galactose residue.

3. The method of claim 2, wherein A is a neuraminic acid residue selected from neuraminic acid or an N-substituted neuraminic acid.

4. The method of claim 2, wherein A is a galactose residue selected from galactose or N-acetylgalactosamine.

5. The method of claim 1, wherein A is enzymatically cleavable from B by a neuraminidase or galactosidase.

6. The method of claim 1, wherein the odorant moiety is derived from an odorant molecule that includes at least one oxygen-containing functional group that is reactive with the anomeric carbon of A.

7. The method of claim 1, wherein the odorant molecule includes at least one ester, aldehyde, ketone and/or hydroxyl functional group that is reactive with the anomeric carbon of A.

8. The method of claim 1, wherein the odorant molecule comprises zingerone, folrosia, vanillin, javanol, methyl diantilis, nonadienol, citronellol, mefresol, anisyl alcohol, cyclohexyl propanol, dihydroeugenol, cinnamyl alcohol, floral pyranol, peony alcohol, geraniol, ionone, ebanol, sandalore, citronellal, benzyl acetone, celery acetone, cetone, claritone, isomuscone, damascone delta, dimethyl octenone, ethyl amyl ketone, exaltone, exaltenone, geranyl acetone, globanone, hedione, jasmatone, jasmone cis, methyl napthyl ketone, methyl undecyl ketone, nerone, plicatone, velvione or vetikone.

9. The method of claim 1, wherein the A carbohydrate is covalently bonded to the B odorant via an —O— linkage.

10. The method of claim 1, wherein the detection of the odor is performed by the subject, or the compound is self-administered, or both.

11. The method of claim 1, wherein the compound is administered in a nasal spray.

12. The method of claim 1, wherein the respiratory pathogen has neuraminidase activity, and the pathogen is a viral pathogen or a bacterial pathogen.

13. The method of claim 1, wherein the viral pathogen is an influenza virus or a parainfluenza virus.

14. The method of claim 12, wherein the bacterial pathogen is Haemophilus influenzae, Streptococcus pneumoniae or Pseudomonas aeruginosa.

15. The method of claim 1, wherein the respiratory pathogen has galactosidase activity, and the pathogen is a bacterial pathogen.

16. The method of claim 15, wherein the bacterial pathogen is Streptococcus pneumoniae.

17. A method of detecting a pathogen with neuraminidase activity or galactosidase activity on a solid surface or in a fluid sample, wherein at least one compound of formula I, II, III or IV is disposed on the solid surface or contained within the fluid sample; the method comprising detecting the presence or absence of an odor by smell, wherein the presence of the odor results from release of the odorant moiety and detects the pathogen on the solid surface or in the fluid sample.

18. The method of claim 17, wherein the solid surface is paper, a test strip, a swab, tissue, wipe, air filter, respiratory mask, clothing, floor, counter, wall, furniture, laboratory equipment, medical equipment, or skin; or the fluid sample is a mucous, saliva, urine, blood, water, detergent, gel, soap or hand sanitizer sample.

19. The method of claim 17, wherein the pathogen has neuraminidase activity, and the pathogen is a viral pathogen of a bacterial pathogen.

20. The method of claim 19, wherein the viral pathogen is an influenza virus or a parainfluenza virus.

21. The method of claim 19, wherein the bacterial pathogen is Haemophilus influenzae, Streptococcus pneumoniae, Pseudomonas aeruginosa or Vibrio cholerae.

22. The method of claim 17, wherein the pathogen has galactosidase activity and the pathogen is a bacterial pathogen.

23. The method of claim 22, wherein the bacterial pathogen is Streptococcus pneumoniae.

24. A method for detecting the presence of Vibrio cholerae in a sample, comprising:
(i) contacting the sample with at least one compound, or a pharmaceutically acceptable salt, ester, hydrate or solvate thereof, comprising the formula A-B, wherein A comprises a carbohydrate that is a neuraminidase or galactosidase substrate, B comprises an odorant moiety, B is covalently bonded to an anomeric carbon of A, and A is enzymatically cleavable from B at the covalent bond site between A and B; and
(ii) detecting the presence or absence of an odor by smell, wherein the presence of the odor results from release of the odorant moiety and indicates Vibrio cholerae in the sample.

25. The method of claim 24, wherein the sample is a water, sewage or fecal sample.

26. A method for detecting a neuraminidase or a galactosidase in a sample, comprising:
(i) contacting the sample with at least one compound, or a pharmaceutically acceptable salt, ester, hydrate or solvate thereof, comprising the formula A-B, wherein A comprises a carbohydrate that is a neuraminidase or galactosidase substrate, B comprises an odorant moiety, B is covalently bonded to an anomeric carbon of A, and A is enzymatically cleavable from B at the covalent bond site between A and B; and
(ii) detecting the presence or absence of an odor by smell, wherein the presence of the odor results from release of the odorant moiety and indicates the presence of a neuraminidase or a galactosidase in the sample.
